(19) United States
(12) Patent Application Publication
(10) Pub. No.: US 2009/0123952 A1
(43) Pub. Date: May 14, 2009

(54) Method of Measuring Amyloid-Beta Peptides

(75) Inventor: John Randall Slemmon, West Windsor, NJ (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignees: PFIZER, INC., New York, NY (US); ELAN PHARMACEUTICALS, South San Francisco, CA (US)

(21) Appl. No.: 11/719,066

(22) PCT Filed: Nov. 14, 2005
(86) PCT No.: PCT/US2005/041029

§ 371 Date: Dec. 3, 2008

Related U.S. Application Data
(60) Provisional application No. 60/627,454, filed on Nov. 12, 2004.

(51) Int. Cl. G01N 33/48; C12Q 1/02

(52) U.S. Cl. 435/7.94; 435/29

(57) ABSTRACT

The present invention relates to methods for measuring Aβ peptides in a sample, particularly a sample of blood, such as whole blood and plasma, and to methods of determining whether a compound alters the amount of Aβ produced by a cell or animal.

### FIELD OF THE INVENTION

The present invention relates generally to assays for quantitative measurement of peptides in a biological sample, and more specifically to quantitative measurement of Aβ in biological fluids.

### BACKGROUND OF THE INVENTION

Aβ is a peptide consisting of variable number of amino acid residues, typically 39-43 amino acids. It was found that, among the Aβ variants or species, Aβ(1-40), Aβ(1-42), and Aβ(11-42) are the major constituents of senile plaques present in the brains of Alzheimer's patients. Other Aβ variants have also been found in the plaques, such as Aβ(3-40), Aβ(3-42), Aβ(4-42), Aβ(6-42), Aβ(7-42), Aβ(8-42), Aβ(9-42), and other shorter variant. (Jan Naslund, et al: Relative abundance of Alzheimer Aβ amyloid peptide variants in Alzheimer disease and normal aging. Proc. Natl. Acad. Sci. USA: vol. 91, pp 8378-8382, 1994). Evidence has shown that Aβ is a small fragment of a much larger protein, referred to as amyloid precursor protein (APP), which is a glycosylated, single-membrane-spanning protein expressed in a wide variety of cells in many mammalian tissues, and arises as a peptide fragment that is cleaved from APP by two proteases called β-secretase and γ-secretase.

Scientific evidence has suggested that overproduction or impaired clearance of Aβ play a seminal role in the formation of AD plaques and pathogenesis of AD and reduction of Aβ plaques has been proposed as a plausible mechanism for the treatment of AD. Consequently, various drug candidates designed to lower amyloid production or enhance its clearance are under investigation. Testing the efficacy of these potential drugs in humans will be greatly facilitated if levels of Aβ peptide in routinely available biological samples, such as blood, could be readily and accurately measured.

The following references reported measurement of Aβ in plasma using standard ELISA: Mehta P. D. et al.: Plasma and cerebrospinal fluid levels of amyloid β proteins 1-40 and 1-42 in Alzheimer disease. Arch Neurol 57, 100-105 (2000); Mehta P. D. et al.: Amyloid β protein 1-40 and 1-42 levels in matched cerebrospinal fluid and plasma from patients with Alzheimer disease. Neuroscience Letters, 304, 102-106 (2001); and Schupf, N. et al.: Elevated plasma amyloid β-peptide 1-42 and onset of dementia in adults with Down syndrome. Neuroscience Letters, 301, 199-203 (2001).

Measurement of Aβ in cellular constituents of blood by using High Performance Liquid Chromatography (HPLC) enrichment in combination with ELISA was reported in Ming Chen, et al.: Platelets are the primary source of amyloid β-peptide in human blood. Biochemical and Biophysical Research Communications, vol. 213, No. 1: 96-03 (1995).

Measurement of Aβ deposited in brains of Alzheimer's disease patients by using HPLC in combination with gel electrophoresis was reported in Kaplan B. and Pras M.: Combined use of micro-preparative gel electrophoresis and reversed-phase high-performance liquid chromatography for purification of amyloid β peptides deposited in brains of Alzheimer's disease patients. J. Chromatography, 769, 363-370 (2002).

An on-line analysis that couples automated reverse-phase chromatography to detection and quantification by mass spectrometry was reported in Clarke, N. J., Crow, F. W., Younkin, S. and Naylor, S.: Analysis of in vivo-derived amyloid-β polypeptides by on-line two-dimensional chromatography-mass spectrometry. Analytical Biochemistry 298, 32-39 (2001).

### SUMMARY OF THE INVENTION

In one aspect, the invention provides quantitative methods of measuring the amount of at least one Aβ species in a sample. The methods involve contacting the sample with a denaturing agent, which result in a sample-denaturing agent mixture, extracting from the sample-denaturing agent mixture a peptide pool comprising the Aβ Species, separating the Aβ species from the peptide pool, and determining the amount of the Aβ species separated from the peptide pool.

In one particular embodiment the invention provides quantitative methods of measuring the amount of at least one Aβ species in a sample of biological fluid, which comprises the steps of contacting the sample with a denaturing agent comprising guanidine hydrochloride; extracting a peptide pool from the sample-denaturing agent mixture by solid phase extraction; separating the Aβ species from the peptide pool by reverse phase HPLC; and determining the amount of the Aβ species separated from the peptide pool by an immunoassay.

In another aspect, the invention provides screening methods for determining whether a compound alters the production of at least one Aβ by a cell. The methods involve administering the compound to a culture comprising the cell; measuring the amount of the Aβ species in a sample from the culture according to a quantitative method of the invention; and comparing the amount of the Aβ species from the culture comprising the cell to which the compound has been administered with the control amount of the Aβ, wherein a difference between amount from the culture and the baseline amount Indicates that the compound alters production the Aβ species by the cell.

In yet another aspect, the invention provides screening methods for determining whether a compound alters the production of at least one Aβ by an animal. The methods involve administering the compound to the animal; obtaining a sample from the animal to which the compound has been administered; measuring the amount of the Aβ species in the sample according to a quantitative method of the invention; and comparing the amount of the Aβ species from the animal to which the compound has been administered with the baseline amount of the Aβ, wherein a difference between the amount from the animal to which the compound has been administered and the baseline amount indicates that the compound alters the production of the Aβ species by the animal.

### BRIEF DESCRIPTION OF THE DRAWINGS

FIG. 1: Panel A: Representative HPLC profile showing separation of synthetic Aβ 1-37 (peak 1), 1-40 (peak 2) and 1-42 (peak 3) using the system and under the conditions described in the Experimental section of this application; Panel B: Representative HPLC profile of guanidine hydrochloride-extracted whole blood and the detection of Aβ species by ELISA; Panel C: Representative HPLC Profile of guanidine hydrochloride-extracted plasma and detection of Aβ species by ELISA.

FIG. 2: Panels A through F: Bar graphs showing recovery of Aβ peptides from guanidine hydrochloride-extracted-whole blood; Panels G through L: Bar graph showing recovery of Aβ peptides from guanidine HCl-extracted plasma.

### DETAILED DESCRIPTION OF THE INVENTION

### I. Quantitative Methods of the Invention

In one aspect, the present invention provides quantitative methods of measuring the amount of at least one Aβ species in a sample. The methods involve contacting the sample with a denaturing agent, which results in a sample-denaturing agent mixture, extracting from the sample-denaturing agent mixture a peptide pool comprising the Aβ species, separating the Aβ species from the peptide pool, and measuring the amount of the Aβ species that has been separated from the peptide pool.

A. Aβ Peptides

The quantitative methods of the invention can be adopted for measuring any Aβ species, and are particularly suitable for measuring Aβ species that are major components of amyloid plaques of Alzheimer patients, such as Aβ1-37, 1-40, and 1-42. Methods of the invention can be adopted for measuring the amount of individual Aβ species, total amount of a plurality of Aβ species, or ratio of a plurality of Aβ species in a sample.

B. Sample Collection and Preparation

The quantitative methods of the invention can be adopted for use with any sample where Aβ is present. The methods, however, are particularly suitable for measuring Aβ in biological samples. Examples of suitable samples include (1) biological fluids such as whole blood, serum, plasma, urine, lymph, and cerebrospinal fluid; (2) blood components, such as plasma, serum, blood cells, and platelets; (3) solid tissues or organs such as brain; and (4) cultures of human or animal cell lines or primary cells, such as primary human neurons, and primary neurons from transgenic mice harboring human APP genes, e.g., cells from a transgenic PDAPP animal (e.g., mouse), as well as a 293 human kidney cell line, a human neuroglioma cell line, a human HeLa cell line, a primary endothelial cell line (e.g., HUVEC cells), a primary human fibroblast line or a primary lymphoblast line (including endogenous cells derived from patients with APP mutations), a primary human mixed brain cell culture (including neurons, astrocytes and neuroglia), or a Chinese hamster ovary (CHO) cell line. Methods of the invention are particularly suitable for measuring Aβ in a sample of blood of a human or non-human animal, such as whole blood, plasma, or a sample containing any blood components in any amounts.

Samples of whole blood can be collected using any suitable methods known in the art. Anticoagulants may or may not be used for blood sample collection depending on whether separation of blood components is required, or any other particular requirements. For example, an anticoagulant may be required for separation of plasma but may not be used when serum is to be separated. Any suitable anticoagulants may be used in blood collection. Illustrative examples of suitable anticoagulant include ethylenediaminetetraacetate (EDTA), heparin, and citrate. Plasma and cellular components may be separated from whole blood using any suitable method known in the art, such as centrifugation and filtration.

Samples collected may be stored for later analysis under suitable storage conditions known in the art. For samples intended to be stored, it is desirable that the samples are quickly frozen upon collection using suitable means, such as dry ice or liquid nitrogen, and kept frozen before being processed for Aβ measurement.

The sample that is to be measured for Aβ is brought into contact with a denaturing agent, which results in a sample-denaturing agent mixture. It is preferred that the denaturing agent is also capable of causing release of Aβ peptides that are bonded to other proteins, peptides, or molecules in the sample. Illustrative examples of suitable denaturing agents include guanidine salts and urea. It is preferred that the denaturing agent is a guanidine salt, such as guanidine hydrochloride (guanidine HCl).

The denaturing agent is generally prepared and used as a solution in a buffer at pH 6-8. The type of buffers that may be used to dissolve the denaturing agent is not restricted. An exemplary buffer suitable for the invention is sodium phosphate at 10 mM and at pH 7.2. The amount of the denaturing agent relative to the amount of the sample contacted is not critical to the present invention as long as it is sufficient to denature the sample and may be readily determined by a person skilled in the art based on various factors such as the specific denaturing agent used, the nature of the sample, and the level of activities of Aβ-degradating enzymes present in the samples. Where a guanidine salt is used as the denaturing agent, the concentration of guanidine salt in the sample-denaturing agent mixture typically ranges from about 4 molar to about 10 molar, preferably from about 5 molar to about 8 molar, and more preferably from about 6 molar to about 7 molar. Where urea is used as the denaturing agent, the concentration of urea is generally at about 6 molar or higher.

It is desirable that the sample is contacted with the denaturing agent immediately after the sample is collected in order to minimize degradation of Aβ in the sample. Where the samples are to be stored for later analysis, the denaturing agent may be added after the sample is taken out of storage. The denaturing agent is thoroughly and quickly admixed with the sample, preferably with the aid of a mixing device, such as mechanical mixing devices commonly used in chemical or biological laboratories, for example a vortex mixer, a blender, or a tissue homogenizer. For samples that contain solid components, such as tissues, whole blood, or blood cell pellets, it is preferable that the solid components are disrupted or broken by subjecting the sample, in the presence of the denaturing agent, to suitable physical or chemical treatment, such as homogenization with a mechanical homogenizer or a sonicator, which hare commonly used in biological laboratories.

C. Extraction of Peptide Pool

After the sample is contacted with the denaturing agent, insoluble components in the sample-denaturing agent mixture, including cellular and tissue fragments and debris are removed from the mixture by conventional means known in the art, such as centrifugation and filtration. The resultant solution, which contains Aβ peptides as well other peptides and is hereinafter referred to as “denatured solution,” is recovered and the Aβ peptides In the denatured solution are extracted. Any extraction methods suitable for extracting peptides may be adapted for use in the methods of the invention. One example of extraction method particularly suitable for use in the invention is solid phase extraction (SPE) using a hydrophobic or reversed-phase matrix. Other formats that use reverse-phase capture and concentration such as serial or parallel reverse-phase chromatography systems can also be used. Prior to subjecting the denatured solution to extraction for the peptides, It is desirable that the denatured solution is diluted with appropriate medium to reduce the overall concentration of the denaturing agent in the mixture. Where a guanidine salt is used as the denaturing agent, it is preferable that the solution is diluted such that the concentration of the denaturing agent in the solution is lowered to approximately 2.5 to 3.5 molar, preferably 3 molar. The final pH of the denatured solution may be adjusted to pH 2-3 with an acid solution, such as 0.5% phosphoric acid in water (v/v). Alternatively, the sample-denaturing agent mixture may be diluted prior to removal of the insoluble components.

In a specific embodiment, peptides In the denatured solution are extracted with reversed SPE. Procedures for extracting peptides with reverse SPE are known in the art, One example of such a method that is suitable for the present invention is described in J Randall Slemmon (Slemmon J R. Hughes C M. Campbell G A. Flood D G. (1994) Increased levels of hemoglobin-derived and other peptides in Alzheimer's disease cerebellum. Journal of Neuroscience. 14:2225-22350, the disclosure of which is incorporated herein by reference. Briefly, the denatured solution is loaded to a reversed SPE device comprising C18 matrix in a SPE cartridge having pore size between 50 and 130 Angstroms, at flow rates of approximately 2 milliliters/minute. The cartridge is equilibrated in 0.1% trifluoroacetic acid in water prior to use. The unbound material is removed by washing the cartridges in appropriate volume, such as 10 milliliters, of the same buffer. Bound peptides are eluted in appropriate volume of a proper eluant, such as 7-10 milliliters of a solution comprising 0.1% trifluoroacetic acid and 70% acetonitrile in water, and recovered. The peptide fractions recovered are then dried by appropriate means, such as in a vacuum system commonly used in a biological laboratory. The peptides that are extracted from the denatured solution are herein collectively referred to as “peptide pool.”

D. Separation of Aβ from the Peptide Pool by HPLC

For preparing for HPLC separation, the dried peptide fractions extracted from the denatured solution are re-dissolved in a suitable medium. Any medium may be used as long as it maintains Aβ peptides in solution and is compatible with the chromatography procedure. One exemplary suitable medium for dissolving the peptide fractions is a solution comprising acetic acid, such as acetic acid in water, at concentrations ranging from 20% to 30%, preferably 25%. The volume of the medium can vary and be adjusted based on various factors known to a person skilled in the art, such as the predicted concentrations of peptides. After the peptides are dissolved in the medium, an appropriate amount of trifluoroacetic acid is added in the solution, with the final concentration of trifluoroacetic acid ranging from 0.06% to 0.10%, and final concentration of acetic acid ranging from 5% to 12%. The sample is then chromatographed by reverse-phase HPLC using a suitable protocol known in the art. For example, the Aβ peptides can be separated by HPLC on a C-18 reverse column, such as Vydac 218TP54 column (4.6×250 mm, 300 Angstrom pore size), using acetonitrile as mobile phase; eluent: A. 0.1% TFA, B. 0.1% trifluoroacetic acid in 80:20 acetonitrile:water; linear gradient from 25 to 55% over 70 minutes at a flow rate of 1.0 milliliters/minute. Mobile phase gradients may be modified In order to optimize the run time and resolution of the different Aβ peptide species. The elution of peptide fractions may be monitored by UV absorbance at 214 nm. It is useful to outfit the injector with a high capacity injection module and integrated fraction collector.

E. Quantification of Aβ Peptides

Quantification of Aβ peptides recovered from the HPLC separation can be accomplished by methods known in the art, such as immunoassay and mass spectrometry.

In one aspect of the invention, immunoassay is employed for the quantification. Immunoassays are immunological detection techniques that employ binding substances, such as antibodies, specific for the peptide or protein to be detected, and are known in the art. Examples of immunoassays include enzyme-linked immunosorbent assay (ELISA), Western blotting, radioimmunoassay, and the like.

The particular Aβ species that are measured from among the group of all Aβ peptides recovered from HPLC separation depends on the particular measuring method used. In the case of using immunoassays, the particular Aβ species detected by such methods depend upon the particular binding substances, such as antibodies, employed. For example, an antibody raised against the junction region of Aβ may detect Aβ whose carboxy termini extend beyond amino acid 40; but it may also detect AD whose amino termini do not extend to amino acid no. 1 of Aβ peptide. Also, an antibody that is raised against amino acids 33-42 of Aβ and does not cross react with Aβ (1-40) will bind to Aβ species ending at amino acids of Aβ 41, 42, and 43. Therefore, determining the specificity of the binding substances will assist in determining exactly which Aβ species are being detected.

In one embodiment, the method to detect Aβ is an immunoassay involving a single antibody that is specific for a particular Aβ species, or a plurality of particular Aβ species from other APP fragments which might be found in the sample, such as a single antibody ELISA methods or radioimmunoassays.

In another embodiment, the method to detect Aβ is an immunoassay Involving two antibodies, in which one antibody is specific for one or more particular Aβ peptides and the other antibody is capable of distinguishing Aβ and Aβ fragments from other APP fragments which might be found in the sample. In particular, it has been found that antibodies which are mono-specific for the junction region of Aβ are capable of distinguishing Aβ from other APP fragments. The junction region of Aβ refers to the region of the Aβ that is centered at amino acid residues 16 and 17, typically spanning amino acid residues 13 to 28. Such “junction-recognizing” antibodies may be prepared using synthetic peptides having that sequence as an immunogen.

A preferred immunoassay technique for detecting Aβ is a two-site or “sandwich” ELISA. This assay employs two antibodies, one of which is a capture antibody, usually bound to a solid phase, and the other a labeled reporter antibody (also called “detection antibody” or “detecting antibody.”) In this method, target Aβ species are captured from the sample by the capture antibody specific for the target Aβ species and the capture of the Aβ species is detected using the labeled reporter antibody specific for Aβ species. For example, total Aβ can be measured using a capture antibody to the junction region and a reporter antibody that should detect virtually all the Aβ species, e.g., an antibody raised against amino acids 1-12 of Aβ. Alternatively, total Aβ can be measured using antibodies that are specific for the junction region, such as amino acids 17-25, as detection antibodies and using antibodies that are specific for amino acids near the amino terminus as capture antibodies. Various sandwich ELISA methods for measuring Aβ are known in the art and can be adopted for use in the present invention. One example of sandwich ELISA for measuring total Aβ, which can be used in the present invention, is described by Johnson-Wood et al, which employs a high affinity capture antibody (antibody 266 raised against amino acids 13-28 of the Aβ sequence) and the biotinylated Aβ amino-terminal-specific antibody 3D6 as the reporter. (Johnson-Wood K. Lee M. Motter R. Hu K. Gordon G. Barbour R. Khan K. Gordon M. Tan H. Games D. Lieberburg I. Schenk D. Seubert P. McConlogue L. Amyloid precursor protein processing and A beta42 deposition in a transgenic mouse model of Alzheimer disease. Proc. Natl. Acad. Sci. U.S.A. 94:1550-1555 (1997). Johnson-Wood et al also described a Aβ1-42-specific sandwich ELISA, which employs the capture antibody mAb 21F12 (Aβ33-42) and biotinylated 3D6 reporter antibody, and a Aβx-42 sandwich ELISA, which uses antibody 266 as the capturing antibody and biotnylated 21F12 as the reporter antibody, both of which methods may be used in the present invention. Other examples of ELISA that can be used In the present invention are set forth in U.S. Pat. No. 6,610,493.

Antibodies specific for Aβ are known in the art and are commercially available. Methods of preparing such antibodies are also known in the art. Antibodies specific for a particular Aβ species may be produced by in vitro or in vivo techniques. In vitro techniques involve exposure of lymphocytes to the immunogens, while in vivo techniques require the injection of the immunogens into a suitable vertebrate host. Suitable vertebrate hosts are non-human, including mice, rats, rabbits, sheep, goats, and the like. Immunogens are injected into the animal according to a predetermined schedule, and the animals are periodically bled, with successive bleeds having improved titer and specificity. The injections may be made intramuscularly, intraperitoneally, subcutaneously, or the like, and an adjuvant, such as incomplete Freund's adjuvant, may be employed.

If desired, monoclonal antibodies can be obtained by preparing immortalized cell lines capable of producing antibodies having desired specificity. Such immortalized cell lines may be produced in a variety of ways. Conveniently, a small vertebrate, such as a mouse, is hyperimmunized with the desired immunogen by the method just described. The vertebrate is then killed, usually several days after the final immunization, the spleen cells removed, and the spleen cells immortalized. The manner of immortalization is not critical. Presently, the most common technique is fusion with a myeloma cell fusion partner, as first described by Kohler and Milstein (1975) Nature 256:495-497. Other techniques including EBV transformation, transformation with bare DNA, e.g., oncogenes, retroviruses, etc., or any other method which provides for stable maintenance of the cell line and production of monoclonal antibodies. Specific techniques for preparing monoclonal antibodies are described in Antibodies: A Laboratory Manual, Harlow and Lane, eds., Cold Spring Harbor Laboratory, 1988. Examples of methods for preparing such antibodies and utilizing such antibodies in an exemplary ELISA are set forth in the U.S. Pat. No. 6,610,493.

### II. Screening Methods of the Invention

In another aspect, the invention provides screening methods for screening compounds that increase or decrease the production of at least one Aβ species by a cell, particularly an Aβ species that is a major component of amyloid plaques in brain of Alzheimer patients. Compounds that decrease production of Aβ are candidates for use in treating the disease, while compounds that increase production of Aβ may hasten the disease and are to be avoided by humans. Screening methods of the invention for determining whether a test compound alters the production at least one Aβ species produced by a cell involve administering the compound to the cell, usually in culture, measuring the amount of the Aβ species produced by the cell using the quantitative methods of the invention described herein above, and determining whether this amount is greater than, less than, or the same as the control amount produced by cell. If the amounts are different, then the compound affects the production of the Aβ by the cell. This amount can be measured, for example, in a sample from the culture, such as medium conditioned by the cell in culture, or in extracts derived from cells harvested from the culture.

The control amount generally will be determined by measuring the Aβ species produced by the cell in the absence of the compound. However, one also may determine the control amount by extrapolation; measuring the amount of the Aβ produced upon administration of different amounts of the compound to the cell, and using these values to calculate the control amount. In certain instances measuring a control amount for the purposes of comparison may not be necessary because the effect of the compound on the Aβ production is evident. For example, a compound may render a given Aβ species undetectable in a cell that normally produces detectable amounts, indicating that the compound decreases the Aβ production from the amount expected in its absence.

In one embodiment, the invention provides screening methods for determining whether a compound alters the production of total Aβ by a cell. The methods involve measuring total Aβ produced by the cell in the presence and absence of the test compound, using the quantitative methods of the invention described herein above.

In another embodiment, the invention provides screening methods for determining whether a compound alters the production of a given Aβ species by a cell to a different degree than it alters the production of total Aβ by the cell. The methods involve administering the compound to the cell, usually in culture. Then, the production of the given Aβ species and production of total Aβ by the cell are determined using a quantitative method of the invention. Then, the productions of the given Aβ species and total Aβ are compared. The comparison indicates whether the compound alters the production of the given Aβ species instead of or in addition to total Aβ.

In a further aspect, the invention provides screening methods for determining whether a compound alters the production or level of at least one Aβ species in an animal, including a human. The methods involve administering the test compound to the animal, collecting a sample from the animal, and measuring the amount of the Aβ in the sample using the quantitative methods of the invention, and determining whether the level of the Aβ is different from the baseline level of the animal. The baseline level generally will be a control level determined by measuring the AD in a sample from an animal in the absence of the compound. The sample for measuring the baseline level may be collected from an animal before the compound is administered to the same animal, or from a control animal that is not subject to dosing with the test compound. However, the baseline level may also be determined by extrapolation by measuring the amount of the Aβ produced upon administration of different amounts of the compound to the animal, and using these values to calculate the baseline level. In certain instances measuring a baseline level for the purposes of comparison may not be necessary because the effect of the compound on the Aβ level is evident. For example, a compound may render a given Aβ species undetectable in a sample from an animal that normally produces detectable amounts, indicating that the compound decreases the Aβ level from the amount expected in its absence.

Various animal models, including transgenic animal models, may be used in the screening methods of the invention. Examples of animal models are described in International Patent Application WO 93/14200, U.S. Pat. No. 5,387,742, and U.S. Pat. No. 6,610,493. These models are useful for screening compounds that alter the production of Aβ in the quantitative methods of this invention for their ability to affect the course of Alzheimer's disease, both to ameliorate and aggravate the condition. Transgenic mammalian models, more particularly, rodent models and in particular murine, hamster and guinea pig models, are suitable for this use.

A particular non-human transgenic animal is one whose cells harbor a PDAPP construct. A PDAPP construct is a nucleic acid construct that comprises a mammalian promoter operatively linked to a cDNA-genomic DNA hybrid coding for the expression of APP. The cDNA-genomic DNA hybrid contains a cDNA sequence encoding APP770 or a cDNA sequence encoding APP770 with a naturally occurring mutation (e.g., a Hardy mutation or the Swedish mutation) substituted with genomic DNA sequences. The genomic DNA sequences consist of exon 6 and an amount of the adjacent downstream intron sufficient for splicing, the KI and OX-2 coding region and an amount of each of their upstream and downstream introns sufficient for splicing, and exon 9 and an amount of the adjacent upstream intron sufficient for splicing, substituted into the corresponding region of the cDNA sequence encoding APP770, or the cDNA encoding APP770 with a naturally occurring mutation. The construct is transcribed and differentially spliced in mammalian cells to form mRNA molecules that encode and that are translated into APP695, APP751 and APP770. In certain embodiments, the construct contains a PDGF-beta promoter operatively linked with a hybrid sequence encoding an APP gene harboring a Hardy mutation (V717F), and the SV40 polyadenylation signal. One version of the PDAPP construct is disclosed in U.S. Pat. No. 6,610,493.

Another useful non-human animal model harbors a copy of an expressible transgene sequence that encodes the Swedish mutation of APP (asparagines 595-leucine 596). The sequence generally is expressed in cells that normally express the naturally-occurring endogenous APP gene (if present). Such transgenes typically comprise a Swedish mutation APP expression cassette, in which a linked promoter and, preferably, an enhancer drive expression of structural sequences encoding a heterologous APP polypeptide comprising the Swedish mutation. Aβ levels can be measured in any body fluid or tissue sample, for example, brain homogenate. The transgenic animals express the Swedish mutation APP gene of the transgene (or homologously recombined targeting construct), typically in brain tissue. Preferably, one or both endogenous APP alleles are inactivated and incapable of expressing the wild-type APP.

The sample in which the level of the Aβ is to be measured can be a sample of any fluids, or solid tissues or organ from the animal or human. It is preferred that the sample is a sample of a fluid, such as whole blood, plasma, serum, urine, lymph, or cerebrospinal fluid, more preferably whole blood.

A test compound can be any molecule, compound, or substance that can be added to the cell culture or administered to the test animal without substantially interfering with cell or animal viability. Suitable test compounds may be small molecules (i.e., molecules whose molecular mass is no more than 1000 Daltons), biological polymers, such as polypeptides, polysaccharides, polynucleotides, and the like. For in vitro assays, the test compounds will typically be administered to the culture medium at such amounts that would result a concentration of the compound in the medium ranging from about 1 nM to 1 mM, usually from about 10 μM to 1 mM. For in vitro assays using whole animals, the test compounds will typically be administered to the animal at a dosage (expressed as amount of the compound per kilogram of body weight of the animal) ranging from 1 ng/kg to 100 mg/kg, usually from 10 pg/kg to 1 mg/kg.

Test compounds that are able to reduce the level of one or more Aβ species, preferably by at least 20%, more preferably by at least 50%, are likely to be beneficial in the treatment of Alzheimer's disease or other Aβ-related conditions.

### III. Definitions

Following are definitions of certain terms as used in the application in describing the invention.

The term “sample” is defined by its ordinary meaning understood by a person skilled in the art and refers to any material in which the presence or amount of Aβ is to be determined by methods of the invention. It can be in any form such as fluids, solids, and tissues. For example, a sample of blood (or a “blood sample”) is a portion of blood taken from an animal and is representative of the blood in the animal body.

The term “Aβ” refers to a family of peptides that are the principal chemical constituent of the senile plaques and vascular amyloid deposits (amyloid anglopathy) found in the brain in patients of Alzheimer's disease (AD), Down's Syndrome, and Hereditary Cerebral Hemorrhage with Amyloidosis of the Dutch-Type (HCHWA-D). Aβ is also known in the art as “amylold beta protein,” “amyloid beta peptide,” “A beta,” “beta AP,” “A beta peptide,” or “Aβ peptide.” In whatever form, Aβ is a fragment of beta-amyloid precursor protein (APP). Aβ comprises variable number of amino acids, typically 39-43 amino acids. The term “Aβ” also refers to related polymorphic forms of Aβ, including those that result from mutations in the Aβ region of the APP normal gene.

The term “Aβ species” or “Aβ variant” refers to an individual Aβ having a particular amino acid sequence. An Aβ species is commonly expressed as “Aβ (x-y)” wherein x represents the amino acid number of the amino terminus of the Aβ and y represents the amino acid number of the carboxy terminus. For example, Aβ(1-43) is an Aβ species or variant, whose amino terminus begin at amino acid number 1 and carboxy terminus ends at amino acid number 43, a sequence of which is:

```
1
Asp Ala Glu Phe Arg His Asp Ser Gly Tyr Glu Val
                15
His His Gln Lys Leu Val Phe Phe Ala Glu Asp Val
                                        30
Gly Ser Asn Lys Gly Ala Ile Ile Gly Leu Met Val
Gly Gly Val Val Ile Ala Thr
```

Examples of other Aβ species includes, but not limited to, (1) Aβ whose amino-terminus begin at amino acid number 1 of Aβ (1-43) shown above and whose carboxy-terminus ends at different amino acid number, such as Aβ(1-39), Aβ(1-40), Aβ(1-41), and Aβ(1-42), (2) Aβ whose amino acid sequences differ from Aβ(1-43) shown above at the amino-terminus or both termini, such as Aβ(11-42), Aβ(3-40), Aβ(3-42), Aβ(4-42), Aβ(6-42), Aβ(7-42), Aβ(8-42), and Aβ(9-42).

The term “total Aβ” refers to a plurality of Aβ species detected in a sample by a given assay wherein individual Aβ species are not discriminated.

The term “whole blood” means blood from a human or animal containing both cellular components and liquid component. Whole blood can be in coagulated state or non-coagulated state. “Whole blood” also includes blood wherein portion or all of the cellular components, such as white blood cells or red blood cells, have been lysed. “Whole blood” also includes blood wherein small amount of the cellular component or liquid component has been removed.

The term “plasma” refers to the fluid component of the whole blood. Depending on the separation method used, plasma may be completely free of cellular components, or may contain various amounts of platelets and/or small amount of other cellular components. Because plasma is serum plus the clotting protein fibrinogen, the term “plasma” is used broadly herein to encompass “serum.”

The term “denaturing agent” refers to a substance or mixture of substances that at a sufficient concentration is capable of inactivating, inhibiting, or otherwise reducing the activity of enzymes that cause degradation of Aβ in a sample.

The term “contacting” or “contact” means bringing together a denaturing agent into physical proximity to a sample the Aβ of which is to be measured.

### IV. Experimental

It is believed that one skilled in the art can, using the preceding description, practice the present invention to its fullest extent. The following detailed examples are merely illustrative and not limitations of the disclosure and claims in any way. Those skilled in the art will promptly recognize appropriate variations.

### Measurement of Total Aβ Peptides in Whole Blood and Plasma

A. Materials

Synthetic Aβ1-38, 1-40 and 1-42 were obtained from Bachem (King of Prussia, Pa.) and were of 95% purity; [¹²⁵I]Aβ1-40 was from Amersham Biosciences (Upsala, Sweden). Mouse IgG resin and other fine chemicals, unless otherwise noted, were obtained from Sigma (St. Louis, Mo.)

B. Sample Collection and Preparation

Blood samples were obtained from non-fasted healthy individuals. Subjects are labeled A-G with the following vital information. A=45 years old, female; B=42 years, female; C=46 years, female; D=46 years, male; E=24 years, male; F=35 years, female and G=58 years, female.

One hundred milliliters of whole blood was collected in 10 EDTA Vacutainer tubes (Becton, Dickinson & Company, Franklin Lakes, N.J.) and three of these were transferred immediately into 50 ml polypropylene centrifuge tubes (Corning) as 5 ml aliquots and frozen on dry ice. Plasma samples and blood pellets were also prepared from four of the remaining tubes by centrifuging 10 mls of whole blood at ca. 1000×g for 15 min at 4° C. (Beckman-Coulter, Fullerton, Calif.). The plasma was carefully aspirated from the cell pellet and frozen in either 5 ml or 1 ml aliquots in the same manner as that used for the whole blood. The cell pellets from the 10 ml blood samples were also immediately transferred to the same type of polypropylene tube and frozen on dry ice. All samples were stored at −70° C. and analyzed within 1 month of collection.

C. Preparation of Neat Plasma prior to Analysis by ELISA

When using non-denatured plasma samples 1 ml aliquots were preabsorbed prior to direct analysis on ELISA in order to lower non-specific background signal. Mouse IgG-agarose resin (1 mg/ml gel, Sigma, St. Louis, Mo.) was washed twice with 10 volumes of PBS pH 7.5, centrifuged for 15 seconds at maximum speed in an Eppendorf micro-centrifuge and then the pellet was added to plasma that had been thawed on wet ice (0.1 ml resin/ml plasma.). The plasma plus resin slurry was rotated (Scientific Industries Inc., Bohemia, N.Y.) for 18 hrs at 4° and the resin was removed by centrifugation as before. The decanted supernatant was diluted 1:3 with Specimen Diluent (10 mM sodium phosphate, pH 7.4, 0.6% human serum albumin, 0.05% Triton X405 and 0.1% thimerosal) containing 2 M sodium chloride (ICN, Costa Mesa, Calif.), yielding a final sodium chloride concentration of 0.5M. This material was used to directly measure Aβ-content in non-denatured plasma using the ELISA (see below).

D. Extraction of Peptide Pool Using Solid-Phase Extraction (SPE)

Five ml aliquots of frozen whole blood, plasma or blood cell pellet were vortexed into 46 ml of 6.5M guanidine hydrochloride, 10 mM sodium phosphate pH 7.2, to thaw and then homogenized using a Polytron (PTA-20S probe) for approximately 30 seconds (Brinkman Instruments Inc., Westbury, N.Y.). The denatured samples were diluted to 3 M guanidine HCl by the addition of 49 ml of 0.5% (v/v) phosphoric acid (final pH=2.5). The samples were then re-homogenized for 30 seconds at low speed and centrifuged at 38,000 g (Beckman-Coulter) for 10 min at 15° in 250 ml bottles in order to eliminate any foam. Only a very small pellet resulted. The supernatant was decanted and re-centrifuged in 100 ml Quick Seal tubes (Beckman-Coulter Inc., Miami, Fla.) at 48,000×g for 30 min at 15°. Another small pellet resulted. Peptides in the supernatant were concentrated and desalted by solid-phase extraction over two C18 SepPak Plus cartridges (Waters, Milford, Mass.) coupled in series. The cartridges had been equilibrated prior to use in 0.1% trifluoroacetic acid in water and unbound material was removed by washing cartridges in the same buffer. Bound peptides were eluted in 0.1% trifluoroacetic acid, 70% acetonitrile in water. Samples were dried in a SpeedVac system (Thermo-Savant, Holbrook, N.Y.). The procedure for the recovery of Aβ from solid-phase extraction was optimized using I-125 labeled Aβ 1-40 peptide in parallel experiments.

E. Reverse-Phase Analysis of Peptides

Peptide fractions were reconstituted in 500 ul of 25% acetic acid, sonicated in a bath sonicator for 30 minutes, diluted with 1 ml of 0.1% trifluoroacetic acid, and sonicated for a further 30 minutes. A small insoluble pellet was discarded after centrifugation in an Eppendorf microcentrifuge at maximum speed, and the supernatant chromatographed by reverse-phase HPLC using an Agilent Technologies' 1100 HPLC system (Agilent Technologies inc., Palo Alto, Calif.) outfitted with a high capacity injection module and integrated fraction collector. Separation of peptide species was carried out on a Vydac 218TP54 column (4.6×250 mm), at a flow rate of 1 ml/min in 0.1% trifluoroacetic acid, using acetonitrile as the mobile phase. A linear gradient from 25 to 55% buffer B (0.1% trifluoroacetic acid, 80% acetonitrile) over 70 minutes was used, followed by isocratic elution with 100% B for 10 min. The elution of peptide fractions was monitored by UV absorbance at 214 nm. One ml fractions were collected and dried overnight in a SpeedVac system. The dried fractions were re-suspended in 500 μl specimen diluent containing 0.5 M sodium chloride each and vortexed for 1 hour. Generally, 100 μl aliquots were assayed for total Aβ using the ELISA. Recovery of Aβ peptide from HPLC was determined by spiking samples from the SPE with I-125 labeled Aβ 1-40 and determining the yield of radioactivity in the chromatographic fractions.

F. Quantification of Total Aβ by ELISA

Quantification of Aβ was conducted using a total Aβ sandwich ELISA as described in Johnson-Wood et al.: Amyloid precursor protein processing and A beta 42 deposition in a transgenic mouse model of Alzheimer disease. Proc. Natl. Acad. Sci. U.S.A. 94:1550-1555 (1997). Briefly, a high affinity capture antibody (antibody 266 raised against amino acids 13-28 of the Aβ sequence) was coated onto 96 well ELISA plates and the biotinylated Aβ amino-terminal-specific antibody 3D6 was used as the reporter. The biotinylated 3D6 antibody and Avidin-horseradish-peroxidase (Vector Laboratories inc., Burlingame, Calif.) were diluted in Specimen Diluent in lieu of casein assay buffer. Antibody binding was monitored with Slow TMB-ELISA HRP substrate (Pierce, Rockford, Ill.) by reacting for 30 minutes, after which color development was stopped with 1 M H₂SO₄. Assay results were quantified in a Spectramax Plus 384 spectrophotometer (Molecular Devices Co., Sunnyvale, Calif.) by measuring the difference in absorbance at 450 nm and 650 nm. Total Aβ peptide from HPLC-separated samples was determined by summing immunopositive peaks. All such peaks eluted between 30 and 60 min.

G. Results

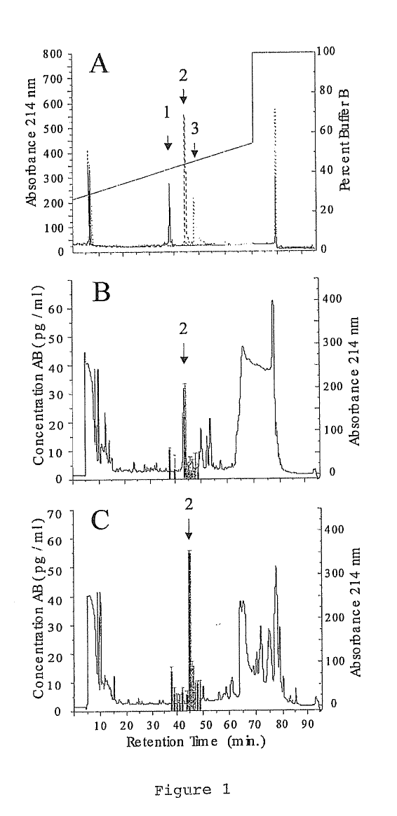

Each step of the extraction and separation process was analyzed for the recovery of Aβ 1-40 by using synthetic peptide labeled with I-125. Recovery from SPE samples were 88.15 (±0.85 s.e.) for blood and 94.55 (±0.36 s.e.) for plasma. Recovery from HPLC was 92.51 (±4.94 s.e.) for blood and 93.91 (±2.14 s.e.) for plasma. This yielded overall recoveries of 81% for blood and 89% for plasma. The results show that the recovery Aβ was sufficiently reproducible to provide a quantifiable process. In addition, it can be observed in FIG. 1A that the HPLC system employed allowed baseline-resolution of Aβ peptides 1-37, 1-40 and 1-42, which is a significant advantage over other methods. Thus, the methods were quantitative for total Aβ and at the same time allow differentiation of these Aβ species.

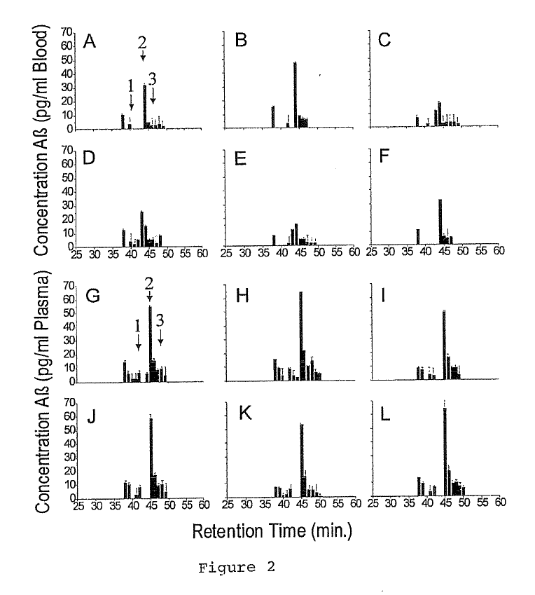

Results of measurements of Aβ in the samples of whole blood and plasma from six human subjects are shown in FIGS. 1 and 2 and Tables 1-3.

Panel A of FIG. 1 shows the separation of synthetic Aβ 1-37 (peak 1), 1-40 (peak 2) and 1-42 (peak 3). The results show that the resolution of the different Aβ species are sufficient to differentiate them by elution time on RP-HPLC. Aβ 1-37 and 1-40 are separated by approximately 6-7 minutes while Aβ 1-40 and 1-42 are separated by 2-3 minutes. Approximately 10 ug of each peptide was injected.

Panel B of FIG. 1 shows a representative HPLC profile of guanidine hydrochloride-extracted whole blood and the detection of Aβ species by ELISA. The bars represent Aβ species quantified from HPLC fractions by ELISA using the anti-Aβ (1-5) and anti-Aβ (17-23) antibodies. Error bars represent standard deviation of two determinations.

Panel C of FIG. 1 shows a representative HPLC Profile of guanidine hydrochloride-extracted plasma and detection of Aβ species by ELISA. The bars represent Aβ species quantified from HPLC fractions. Error bars are standard deviation of two determinations. The elution position of Aβ 1-40 was confirmed by detecting 1-125-labelled peptide and is indicated in panels B and C as peak 2.

Panels A through F of FIG. 2 shows recovery of Aβ peptides from guanidine hydrochloride-extracted-whole blood. Aβ species in each HPLC fraction were quantified by ELISA. The relative position of Aβ 1-40 (2) was determined using a radioactively-labeled Internal standard in a parallel separation and the position of Aβ 1-37 and 1-42 were estimated based upon the separation shown in FIG. 1. Error bars are standard deviation of two determinations.

Panels G through L of FIG. 2 shows recovery of Aβ peptides from guanidine HCl-extracted plasma. Panels G-L correspond respectively to the whole blood of subjects in panels A through F. Aβ species in each HPLC fraction were quantified by ELISA. The bars represent Aβ species quantified from HPLC fractions. Error bars are standard deviation of two determinations.

Table 1 shows total Aβ measured in plasma in its native state (i.e without contact with a denaturing agent) and in plasma that was contacted with a denaturing agent (guanidine HCl).

Table 2 shows total Aβ measured from plasma and whole blood, both of which were contacted with a denaturing agent (guanidine HCl).

Table 3 shows total Aβ measured from plasma and resultant cell pellets, both of which were contacted with a denaturing agent (guanidine HCl).

TABLE 1
Table 1: Comparison of Aβ measured from native or denatured
plasma: ELISA for total Aβ was performed either with or without
first contacting the sample with 6 M guanidine HCl.

| | | | | Non- | |
|---|---|---|---|---|---|
| | Non-Denatured | | Denatured | Denatured: | |
| | Plasma (native) | | Plasma | Denatured | T-Test |
| Subject | (pg/ml) | | (pg/ml) | (%) | (p value) |
| A | 82.1 | (±14.4) | 129.7 (±7) | 63.3 | ** 3.0 10⁻⁷ |
| B | 154.8 | (±9.2) | 165.9 (±3) | 93.3 | 1.1 10⁻¹ |
| C | 47.5 | (±14.5) | 110.2 (±4) | 43.1 | ** 6.2 10⁻⁶ |
| D | 68.8 | (±18.9) | 134.8 (±3) | 51.0 | ** 8.9 10⁻⁵ |
| E | 37.5 | (±9.9) | 107.8 (±5) | 34.8 | ** 2.5 10⁻⁸ |
| F | 32.7 | (±10.7) | 149.5 (±9) | 21.9 | ** 4.3 10⁻¹² |

Denatured plasma was enriched on SPE and HPLC prior to assay. Error is standard deviation from either 21 determinations (Non-Denatured Plasma) or 2 determinations (Denatured Plasma). Both types of analysis used plasma from the same bleed.
** denotes a highly significant difference in Aβ peptide levels between non-denatured and denatured plasma. T-test was carried out with Origin Statistical Graphics Software using a 2-way independent paradigm.

TABLE 2
Table 2: Comparison of total Aβ peptides
detected from plasma and whole blood.

| | | | | Plasma: | |
|---|---|---|---|---|---|
| | Plasma | Whole Blood | | Whole Blood | T-Test |
| Subject | (pg\1 ml) | (pg/2 ml) | | (%) | (p value) |
| A | 129.7 (±7) | 125.3 | (±3) | 104.0 | 0.591 |
| B | 165.9 (±3) | 181.2 | (±12) | 91.6 | 0.328 |
| C | 110.2 (±4) | 114.2 | (±18) | 96.5 | 0.847 |
| D | 134.8 (±3) | 175.5 | (±4) | 76.8 | **0.011 |
| E | 107.8 (±5) | 112.0 | (±17) | 96.2 | 0.842 |
| F | 149.5 (±9) | 120.8 | (±9) | 123.8 | 0.149 |

All samples were denatured by contacting the sample with 6 M guanidine HCl. Concentrated samples from SPE were resolved on reverse-phase HPLC. Plasma samples were prepared from the whole blood analyzed in this study. Error is standard deviation of two determinations.
**denotes a highly significant difference in the data sets between analysis of non-denatured and denatured plasma. T-test was carried out with Origin Statistical Graphics Software using a 2-way independent paradigm. This comparison assumed plasma volume of 50% of whole blood (i.e. 2 ml whole blood yields 1 ml plasma) in order to estimate the amount of Aβ peptide recovered in the plasma fraction.

TABLE 3
Table 3: Measurement of Aβ in plasma and resultant
cell pellets: Plasma and cell pellets were separated from
whole blood collected from subjects A, C, D, E and F.

| | Plasma | |
|---|---|---|
| | A-Beta Concentration | Blood Cell Pellet |
| Subject | (pg/ml) | A-Beta Concentration (pg/ml) |
| A | 158.5 ± 9.5 | B.D. |
| C | 137.9 ± 8.5 | B.D. |
| D | 136.5 ± 10.6 | 7.7 ± 1.2 |
| E | 181.6 ± 54.4 | 14.7 ± 1.6 |
| F | 134.7 ± 13.7 | B.D. |

After decanting the plasma, the cell pellet was placed on dry ice. Samples were denatured in 6 M guanidine HCl and desalted on solid-phase extraction (SPE). Concentrated samples from SPE were resolved on reverse-phase HPLC. Aβ was determined on HPLC fractions. Error is standard deviation from duplicate determinations.
B.D. = below detection.

What is claimed is:

1. A method of measuring the amount of at least one Aβ species in a sample, comprising the steps of:
(a) contacting the sample with a denaturing agent, resulting in a sample-denaturing agent mixture;
(b) extracting a peptide pool containing the Aβ species from the sample-denaturing agent mixture;
(c) separating the Aβ species from the peptide pool; and
(d) determining the amount of the Aβ species separated from the peptide pool.

2. The method of claim 1, wherein the denaturing agent comprises a guanidine salt.

3. The method of claim 2, wherein the guanidine salt is guanidine hydrochloride.

4. The method of claim 3, wherein the concentration of guanidine hydrochloride in the sample-denaturing agent mixture is from about 3.0 molar to about 6.5 molar.

5. The method of claim 4, wherein the concentration of guanidine hydrochloride in the sample-denaturing agent admixture is from about 3.5 molar to about 6.0 molar.

6. The method of claim 3, wherein extracting a peptide pool is carried out by solid-phase extraction.

7. The method of claim 3, wherein separating Aβ from the peptide pool is carried out by high performance liquid chromatography.

8. The method of claim 7, wherein in step (c) the peptide pool is dissolved in an aqueous solution comprising acetic acid.

9. The method of claim 3, wherein the amount of the Aβ is measured by an immunoassay.

10. The method of claim 9, wherein the immunoassay is a sandwich ELISA.

11. The method of claim 5, wherein the sample is a sample of a biological fluid.

12. The method of claim 11, wherein the biological fluid is selected from whole blood, serum, plasma, urine, lymph, or cerebrospinal fluid.

13. The method of claim 12, wherein the biological fluid is whole blood.

14. The method of claim 3, wherein the sample is a sample of a tissue or organ from an animal, cell culture, tissue culture, or organ culture.

15. A method of measuring the amount of at least one Aβ species in a sample, comprising the steps of:
(a) contacting the sample with a denaturing agent comprising guanidine hydrochloride, which results in a sample-denaturing agent mixture;
(b) extracting a peptide pool from the sample-denaturing agent mixture by solid phase extraction;
(c) separating the Aβ species from the peptide pool by reverse phase HPLC; and
(d) determining, by an immunoassay, the amount of the Aβ species separated from the peptide pool.

16. A method for identifying a compound that alters the production of at least one Aβ species produced by a cell, comprising the steps of:
(a) administering the compound to a culture comprising the cell;
(b) measuring the amount of the Aβ species in a sample from the culture according to the method of claim 1;
(c) comparing the amount of the Aβ species from the culture comprising the cell to which the compound has been administered with the control amount of the Aβ species; wherein a difference between amount from the culture and the control amount indicates that the compound alters the production of the Aβ species produced by the cell.

17. The method of claim 16, wherein the amount of the Aβ in a sample from the culture is measured according to the method of claim 15.

18. The method of claim 16, wherein the culture comprises primary human neurons or primary neurons from a transgenic animal harboring the PDAPP construct.

19. The method of claim 16, wherein the culture comprises a 293 human kidney cell line, a human neuroglioma cell line, a human HeLa cell line, a primary endothelial cell line, a primary human fibroblast line, a primary lymphoblast line, human mixed brain cells, or a Chinese hamster ovary cell line.

20. A method for Identifying a compound that alters the production of at least one Aβ species by an animal, comprising the steps of:
(a) administering the compound to the animal;
(b) obtaining a sample from the animal;
(c) measuring the amount of the Aβ species in the sample according to the method of claim 1; and
(d) comparing the amount of the Aβ species from the animal to which the compound has been administered with the baseline amount of the Aβ; wherein a difference between the amount from the animal to which the compound has been administered and the baseline amount indicates that the compound alters the level of the Aβ species produced by the animal.

21. The method of claim 20, wherein the animal is a human.

22. The method of claim 20, wherein the animal is a non-human animal.

23. The method of claim 22, wherein the animal is a non-human transgenic animal.

24. The method of claim 23, wherein the cells of the transgenic animal harbor a PDAPP construct.

25. The method of claim 23, wherein the cells of the transgenic animal harbors a copy of an expressible transgene sequence that encodes the Swedish mutation of APP.
